(19) United States
(12) Patent Application Publication
(10) Pub. No.: US 2008/0064970 A1
(43) Pub. Date: Mar. 13, 2008

(54) 12 Lead Ecg Fabric Electrode Belt System

(76) Inventor: Jean-Francois Montplaisir, Quebec (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/576,765

(22) PCT Filed: Oct. 7, 2005
(86) PCT No.: PCT/CA05/01575

§ 371 Date: Apr. 5, 2007

Related U.S. Application Data
(60) Provisional application No. 60/616,627, filed on Oct. 8, 2004.

(51) Int. Cl. A61B 5/0402

(52) U.S. Cl. 600/509

(57) ABSTRACT

There is described a wearable 12-lead ECG fabric electrode belt system having the standard 10 electrodes permitting the capture of a 12-lead ECG by using predetermined electrical vectors passing through the heart. This system is devoid of the electrodes normally present at the feet or lower pelvis area of the patient. Non-adhesive electrodes are used, which is an advantage for the skin of the patient and allows the patient the possibility of removing and repositioning the belt system if need be. All of the electrodes on the chest area are provided in a single line and positioned just below the pectoral line.

### CROSS-REFERENCE TO RELATED APPLICATIONS

This application claims priority of U.S. Provisional Patent Application No. 60/616627, filed on Oct. 8, 2004, which is hereby incorporated by reference.

### FIELD OF THE INVENTION

The present invention relates to the field of monitoring heart activity, and in particular to wearable or portable electrocardiogram monitors.

### BACKGROUND OF THE INVENTION

An electrocardiogram (ECG/EKG) is an electrical recording of the heart that is used in the investigation of heart disease. Cardiologists have confirmed the urgent need for devices that can be worn for a long period to provide an ECG covering more than twenty-four hours. The idea is to enable the observation of cardiac events that are not regularly present in heart activity.

Cardiac contractions are the result of a well orchestrated electrical phenomenon called depolarization. Cell membranes move from their negative resting potential to a more positive threshold which ultimately stimulates them to contract. In the myocardium there are specialized fibers that are very conductive and allow the rapid transmission of electrical impulses across the muscle, telling them to contract. In order to maximize the force of the contraction there is uniformity in the sequence. That is, the atria contract, then the ventricles contract. This allows both sets to fill properly before ejecting the blood to its next destination. These two sections are independent, yet linked to a single impulse, (in a healthy heart,) initiated by the sinoatrial, (or sinus) node. The tissue around the valves helps to channel the impulse from the sinus node through another collection of specialized tissue, the atrioventricular node, that is situated between the two sets of chambers. This area allows slightly slower transmission of the impulse to the ventricles, allowing the atria to empty into the ventricles before they contract and force the blood to the lungs or body. This area, the A/V Node, slows the impulse down to about one twenty-fifth of the original signal then passes it through to the atrioventricular bundle, or the bundle of His. This bundle divides itself into two distinct tracts through the ventricles, the bundle branches, and on to the Purkinje fibers, where the muscle of the ventricle is stimulated to contract from the bottom up, maximizing the force of ejection.

An electrical current in the direction towards the positive end of a bipolar electrode causes a positive deflection of the stylus of the ECG. If the number of myocardial cells (dipoles) in this direction increases, the current will increase as well. The greater the current, the more positive the voltage. An electrical current in the direction away from the positive end of a bipolar electrode causes a negative deflection of the stylus of the ECG. If the number of myocardial cells (dipoles) in this direction increases, the current will increase as well. The greater the current, the more negative the voltage.

Normal ECG captation or HOLTER recordings are made with devices of different sizes which generally use self-adhesive electrodes or suction cups. These electrodes are generally made for short term ECG captation and are not suitable for long term analysis.

12 Lead ECG electrode positioning is a very complexe science. Since these 12 leads are very specific heart electrical activity vectors, and since the mixing of 2 or more electrodes is needed to get one lead, the position of the electrodes must be at least in the same tri-dimensional vector angle to be sure to get a signal as accurate as possible.

In view of the need for long term ECG recordings, and the need to provide flexibility to those who will be wearing an ECG system for a long period of time, there is a need for a simpler way of placing and repositioning the electrodes on the patient.

### SUMMARY OF THE INVENTION

A wearable 12-lead ECG fabric electrode belt system is provided having the standard 10 electrodes permitting the capture of a 12 lead ECG by using predetermined electrical vectors passing through the heart. This system is devoid of the electrodes normally present at the feet or lower pelvis area of the patient. Non-adhesive electrodes are used, which is an advantage for the skin of the patient and allows the patient the possibility of removing and repositioning the belt system if need be. All of the electrodes on the chest area are provided in a single line and positioned just below the pectoral line. The elasticity of the strap provides an automatic adjustment of the distances between the electrodes in order to accommodate patients of varying sizes.

In accordance with a first broad aspect of the present invention, there is provided a belt system for collecting electrocardiogram signals, the system comprising: a chest strap having attachment means for strapping around a chest of a patient, and eight non-adhesive electrodes mounted thereon, aligned and spaced apart on the chest strap at pre-determined positions; a first arm strap having attachment means for strapping around a first arm or shoulder of the patient, and one non-adhesive electrode mounted thereon; and a second arm strap having attachment means for strapping around a second arm or shoulder of the patient, and one non-adhesive electrode mounted thereon.

In accordance with a second broad aspect of the present invention, there is provided a method for collecting electrocardiogram signals on a patient, the method comprising: aligning eight non-adhesive electrodes in a substantially straight line underneath a pectoral line of said patient, said eight non-adhesive electrodes being placed at pre-determined positions; positioning a single non-adhesive electrode on each arm or shoulder of said patient; and collecting bio-electrical signals from heart activity using said electrodes and sending them to a device for storage or analysis.

### BRIEF DESCRIPTION OF THE DRAWINGS

Further features and advantages of the present invention will become apparent from the following detailed description, taken in combination with the appended drawings, in which:

FIG. 1 is a front view of the electrodes and the belts on a patient;

FIG. 2 is a side view of the electrodes and the belts; and

FIG. 3 is a schematic view of how the belts are assembled.

### DETAILED DESCRIPTION OF THE PREFERRED EMBODIMENT

The present invention is for a full ECG monitoring device which collects the ECG data from the patient and transmits it. Acquired data is then processed as per standard 12-Lead ECG apparatus.

The ECG system can be used in conjunction with a data receiver which is a wireless portable device which can be worn on the patient's belt, in his pocket or even in a bag that he is carrying. The data receiver can be connected to a computer, a hand-held PC, a PALM™ Pilot, a cellular or any other device which is compatible with the RS-232 protocol. The acquired data can then be displayed on a small matrix screen of the data receiver and/or on the screen of the computer. A plurality of filters are used on the acquired data to enhance the clarity of the ECG curve obtained and to extract precise information on the patient's heart.

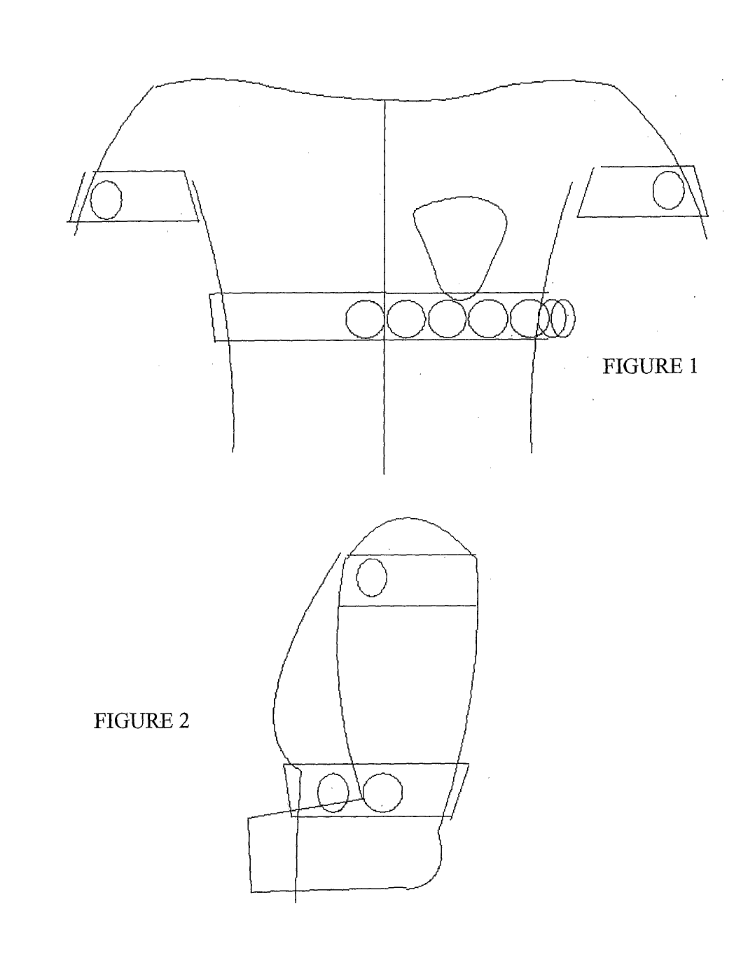

The wearable 12-lead ECG fabric electrode belt system illustrated in FIG. 1 comprises a belt system having the standard 10 electrodes permitting the capture of a 12 lead standard ECG by using predetermined electrical vectors passing through the heart. This system is made of three parts: the left arm electrode belt, the right arm electrode belt and the chest electrode belt made of 8 independent electrodes.

The left arm electrode belt comprises an electrode to be placed on the left arm or shoulder and a belt to retain the electrode. The electrode is connectable to any normal input of an ECG machine.

The right arm electrode belt comprises an electrode to be placed on the right arm or shoulder and a belt to retain the electrode. The electrode is connectable to any normal input of an ECG machine.

The chest electrode belt comprises eight electrodes which are placed in a substantially straight line at pre-determined positions. A belt retains the electrodes. The electrodes are connectable to any normal input of an ECG machine.

As illustrated in FIGS. 1 and 2, the chest belt is placed just under the pectorals. The right and left arm belts are placed on the arm or shoulder. You can see that the LF electrodes must be placed just where the back begins.

This system is intended to be used with portable 12 lead ECG recorders or personal wearable cardiac alarm systems. The device collects bio-electrical signals from heart activity and sends them to a device for storage or analysis.

The chest belt preferably has the following parts
Two 1½″×16″ elastic fabric straps
1″×16″ elastic fabric strap
Two adjustable strap binders
Velcro™ fabric
Eight snap fasteners
Electrical shielded wires
Eight foam squares (1″×1″×⅜″)
Conductive textile (33% silver)
Two mini-USB gold plated connectors
One ten pin connector

To build the belt, the foam squares are fixed on the first 1½″ elastic fabric strap and then covered by the conductive textile, and snapped on the other side of the elastic fabric strap using the fasteners. An electrical wire is then soldered on the snap fastener. A conductive contact should be made between the body side of the conductive fabric and the other side of the wire. Repeat this 8 times and leave ½″ between each electrodes.

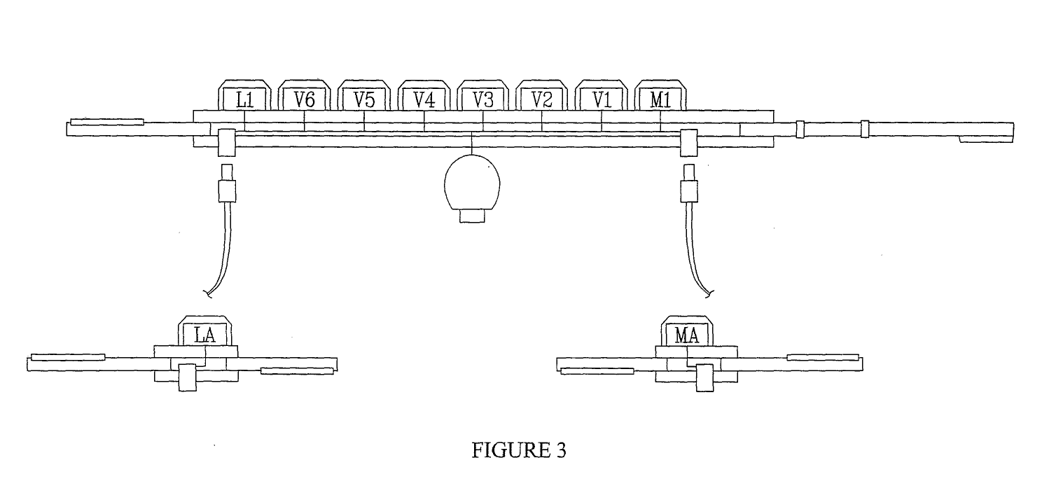

Fix the Velcro™ fabric on the 1″ elastic fabric strap, to be able to strap the belt on the body. Add the two mini-USB using two wires for the arm electrodes. The two-sided wires or the shield should be connected to the RL electrode (FIG. 3). Add the ten pin connector and connect all wires to it. Fix the second 1½″ elastic fabric strap to cover the wires.

Each one of the arm belts preferably have the following components
Two 1½″×2½″ elastic fabric straps
1″×4″ elastic fabric strap
One adjustable strap binder
Velcro™ fabric
One snap fastener
Electrical shielded wired
A foam square (1″×1″×⅜″)
Conductive textile (33% silver)
One mini-USB gold plated connector

The arm belts are constructed as follows : The foam square is fixed on the first 1½″ elastic fabric strap and then covered by the conductive textile, and snapped on the other side of the elastic fabric strap using the fastener. An electrical wire is then soldered on the snap fastener. A conductive contact should be made between the body side of the conductive textile and the other side of the wire.

Fix the Velcro™ fabric on the binder and the 1″ elastic fabric strap, to be able to strap the belt on the body. Add the mini USB connector using two wires for the arm electrodes. Fix the second 1½″ elastic fabric strap to cover the wires.

As is shown in FIG. 3, the a ten-pin connector is hung between the eight electrodes on the chest belt. The ten pin connector is connected to each of the electrodes and may lie on the patient's chest. This single connector is then connected to a standard ECG device used to analyze the captured signals. Alternatively, each electrode may individually be connected to the ECG device.

The belt system can be installed by following simple instructions. This belt system is intended to be put in place by a nurse or physician but can also be put in place by the user himself or a non-medical aid.

Since the belts contain non-adhesive electrodes, they can be worn for long periods of time without creating skin problems. The alignment of all the chest electrodes on a single line also create a real ergonomic advantage, making possible their wearing all day long in everyday activities without refraining from any activity or interfering with the human body movements.

It will be understood that numerous modifications thereto will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

1. A belt system for collecting electrocardiogram signals, the system comprising:
a chest strap having attachment means for strapping around a chest of a patient, and eight non-adhesive electrodes mounted thereon, aligned and spaced apart on said chest strap at pre-determined positions;
a first arm strap having attachment means for strapping around a first arm or shoulder of said patient, and one non-adhesive electrode mounted thereon; and
a second arm strap having attachment means for strapping around a second arm or shoulder of said patient, and one non-adhesive electrode mounted thereon.

2. A belt system as claimed in claim 1, wherein said eight non-adhesive electrodes are spaced half an inch apart.

3. A belt system as claimed in claim 1, wherein each of said electrodes comprise a foam square covered by a conductive textile and a wire soldered to said conductive textile.

4. A belt system as claimed in claim 1, wherein said chest strap comprises two 1½″×16″ straps and said eight electrodes are mounted in between said two 1½″×16″ straps.

5. A belt system as claimed in claim 4, wherein said first arm strap and said second arm strap each comprise two 1½″×2½″ straps.

6. A belt system as claimed in claim 5, wherein each strap is made of an elastic fabric.

7. A belt system as claimed in claim 1, wherein said attachment means are hook and loop fasteners.

8. A belt system as claimed in claim 1, wherein said electrodes are mounted on said straps by snap fasteners.

9. A belt system as claimed in claim 1, further comprising a ten pin connector connected to all of said electrodes and attached to said chest belt, said ten pin connector being connectable to a device used to analyse said signals.

10. A method for collecting electrocardiogram signals on a patient, the method comprising:
aligning eight non-adhesive electrodes in a substantially straight line underneath a pectoral line of said patient, said eight non-adhesive electrodes being placed at pre-determined positions;
positioning a single non-adhesive electrode on each arm or shoulder of said patient; and
collecting bio-electrical signals from heart activity using said electrodes and sending them to a device for storage or analysis.

11. A method as claimed in claim 10, further comprising connecting all of said non-adhesive electrodes into a single ten-pin connector, and connecting said ten-pin connector to a standard electrocardiogram apparatus.

12. A method as claimed in claim 10, wherein said aligning comprises spacing each of said eight non-adhesive electrodes half an inch apart.

13. A method as claimed in claim 10, wherein said aligning comprises mounting said eight non-adhesive electrodes on an elastic fabric strap.

14. A method as claimed in claim 13, wherein said positioning a positioning comprises mounting each of said single non-adhesive electrode on an elastic fabric strap.

15. A method as claimed in claim 14, wherein each elastic fabric strap comprises a hook and loop fastener to secure onto said patient.
